(19) United States
(12) Patent Application Publication
(10) Pub. No.: US 2023/0390352 A1
(43) Pub. Date: Dec. 7, 2023

(54) COMPOSITION COMPRISING SALVIA MILTIORRHIZA EXTRACT AS ACTIVE INGREDIENT FOR PREVENTION AND TREATMENT OF DEPRESSION IN MENOPAUSAL WOMEN

(71) Applicant: UNDBIO.CO.,LTD, Uijeongbu-si, Gyeonggi-do (KR)

(72) Inventors: Se-Chan KANG, Yongin-si (KR); Yang-Mi HER, Suwon-si (KR); Hee-Ju SUH, Yongin-si (KR); Jeong-Eun KWON, Suwon-si (KR)

(73) Assignee: UNDBIO.CO.,LTD, Uijeongbu-si, Gyeonggi-do (KR)

(21) Appl. No.: 18/026,261

(22) PCT Filed: Nov. 17, 2020
(86) PCT No.: PCT/KR2020/016151

§ 371 Date: Mar. 14, 2023

(30) Foreign Application Priority Data
Jan. 22, 2020 (KR) 10-2020-0008707

(51) Int. Cl. A61K 36/537; A61P 25/24

(52) U.S. Cl. CPC A61K 36/537; A61P 25/24; A61K 2236/333

(57) ABSTRACT

The present invention relates to a composition comprising a Salvia miltiorrhiza extract for prevention and treatment of depression in menopausal women. The Salvia miltiorrhiza extract of the present invention is characterized by regulating the release of luteinizing hormone and follicle-stimulating hormone and increasing the release of serotonin and norepinephrine to prevent, alleviate, and treat the hormonal change and depression occurring after menopause. In addition, since the raw material is highly stable, the composition comprising a danshensu-containing Salvia miltiorrhiza extract as an active ingredient retains the efficacy during long-term storage and thus can be suitable for use as an industrial substance. Therefore, the composition comprising a Salvia miltiorrhiza extract as an active ingredient according to the present invention can control hormones in menopausal women and thus can be used as a pharmaceutical composition for prevention and treatment of depression in menopausal women or as a health functional food composition for prevention and alleviation of depression in menopausal women.

### CROSS REFERENCE TO RELATED APPLICATIONS

This Application is a National Stage of International Application No. PCT/KR2020/016151 filed Nov. 17, 2020, claiming priority based on Korean Patent Application No. 10-2020-0008707 filed Jan. 22, 2020, the entire disclosures of which are incorporated herein by reference.

### INCORPORATION BY REFERENCE OF SEQUENCE LISTING

The content of the electronically submitted sequence listing, file name: Q285145_substitute sequence listing as filed; size: 809 bytes; and date of creation: Aug. 15, 2023, filed herewith, is incorporated herein by reference in its entirety.

### TECHNICAL FIELD

The present disclosure relates to a composition for prevention and treatment of depression in menopausal women including a Salvia miltiorrhiza extract as an active ingredient, and more particularly, to a composition for prevention and treatment of depression in menopausal women characterized by regulating the release of luteinizing hormone (LH) and follicle-stimulating hormone (FSH) and increasing the release of serotonin and norepinephrine to prevent, alleviate, and treat the hormonal change and depression occurring after naturally-occurring menopause.

### BACKGROUND ART

Generally, menopausal transition, menopause, and postmenopause are generally referred to as the climacteric, and during this climacteric, female hormones are changed, and particularly, it is characterized that the level of estrogen as a female hormone is reduced. Causes of the menopause largely include natural menopause and induced menopause. The natural menopause refers to amenorrhea that lasts for one year or more without a specific cause, and most often occurs around the age of 50 and is one of the aging phenomena. The induced menopause is caused by surgical menopause, which is subjected to removal surgery of the ovaries, and anthropomorphic removal, which removes the function of the ovaries using radiation or chemotherapy. In addition, the menopause also occurs due to stress, low body weight, chromosomal abnormalities, autoimmune diseases, gene mutations, and the like.

Meanwhile, menopausal symptoms include vaginal dryness, which is a genitourinary symptom, and hot flush, a representative vasomotor symptom, is known to be a major cause of menopausal women visiting hospitals. In addition, it has been reported that not only psychophysiological symptoms such as sleep disorders, depression, and anxiety appear, but also the risk of chronic diseases such as cardiovascular disease and osteoporosis increases with menopause.

Female hormones stimulate the muscles in the uterus to develop, but in the case of menopausal adult women who lack estrogen, the uterine tissue begins to degenerate and eventually even loses its reproductive function. Female sex hormones are mainly related to reproductive activity so that their release is regulated according to the menstrual cycle, and at this time, follicle-stimulating hormone (FSH) is directly responsible for the regulatory action, and when estrogen is released, the release of follicle-stimulating hormone decreases. In other words, as mechanisms of menopausal symptoms, the hormonal change, actions on the central nervous system, and the like have been proposed. During the perimenopause, changes occur in the concentrations of estradiol, follicle-stimulating hormone, progesterone, and inhibin, gonadal steroids regulate mood-regulating neurotransmitters or alter sensitivity, and steroid receptors affect the activity of the central nervous system by acting in the glandular nucleus, amygdala, and hippocampus. That is, in the perimenopause, ovarian follicles are rapidly lost, which leads to an increase in follicle-stimulating hormone to cause a decrease in estrogen. In addition, the estrogen is involved in the production and regulation of neurotransmitters such as acetylcholine (ACh), serotonin, norepinephrine, gamma-aminobutyric acid (GABA), endogenous opiates, dopamine, glutamate, and monoamine oxidase, which is associated with the regulation of a serotonin function by estrogen.

Recently, studies on regulation of follicle-stimulating hormone instead of regulation of estrogen in female menopausal treatment have been actively conducted. That is, there is a research result that follicle-stimulating hormone, which is abnormally increased after menopause, accelerates aging, and it is known to promote osteoporosis, destruction of both ends of the femur, and senile obesity.

Recently, studies on alternative therapies using active ingredients derived from natural products such as herbal medicines and foods capable of regulating the release of follicle-stimulating hormone as well as regulating female hormones without other side effects have been researched and various extract preparation methods have been developed. However, in addition to female hormone substitutes, there is still lacking in the development for natural pharmaceutical compositions or raw materials thereof having excellent effects on suppressing the release of follicle-stimulating hormone and alleviating depression and having fewer side effects than existing synthetic pharmaceutical compositions.

Meanwhile, Salvia miltiorrhiza Bunge (SM) is a medicinal herb that has been widely used in oriental medicine since ancient times and a perennial plant belonging to the dicotyledoneae, Tubiflorae, Labiatae and is native to China. In Salvia miltiorrhiza, roots dried in the sun after removing the fibrous roots are used for medicinal purposes and are collected from the early November to the early March of next year, and the roots dug up in the early November are known to be best. As ingredients contained in the roots of Salvia miltiorrhiza, there has been reported cryptotanshinone, danshensu, isotanshinone I, isotanshinone II, tanshinone I, salvianolic acid B, tanshinone HA, tanshinone IIB, dihydrotanshinone, methyl tanshinonate, methylene tanshinquinone, betasitosterol, hydroxytanshinone, neotanshinone A·B·C, salviol, isocryptotanshinone, miltirone, tanshinol I, tanshinol II, vitamin E, and the like. In addition, Salvia miltiorrhiza has been reported to have effects such as ischemic damage protection, coronary artery relaxation, atherosclerosis inhibition, antihypertensive, antihyperlipidemia, antidiabetic, antibacterial, antioxidant, anticancer, antimutagenic, and antithrombotic. Research on various uses of a Salvia miltiorrhiza extract using these functionalities has been conducted, but studies on effects of regulating the release of follicle-stimulating hormone and alleviating depression among menopausal symptoms have not yet been conducted.

Therefore, in order to increase the usability of Salvia miltiorrhiza, the present inventors have continuously repeated research on Salvia miltiorrhiza, and as a result, confirmed that a Salvia miltiorrhiza extract had an effect of alleviating depression by regulating follicle-stimulating hormone in menopausal women and regulating serotonin and norepinephrine, and then completed the present disclosure. That is, accordingly, the present disclosure can be used as a composition including a Salvia miltiorrhiza extract as an active ingredient for prevention and treatment of depression in menopausal women.

### DISCLOSURE

### Technical Problem

An object of the present disclosure is to provide a pharmaceutical composition for prevention and treatment of depression in menopausal women, including a Salvia miltiorrhiza extract as an active ingredient.

Another object of the present disclosure is to provide a composition for regulating the release of female hormones, including a Salvia miltiorrhiza extract as an active ingredient.

Yet another object of the present disclosure is to provide a health functional food composition for prevention and alleviation of depression in menopausal women, including a Salvia miltiorrhiza extract as an active ingredient.

The various problems to be solved in the present disclosure described above are not limited thereto, but may be clearly understood by those skilled in the art through the details described in embodiments and claims to be described later.

### Technical Solution

One aspect of the present disclosure provides a pharmaceutical composition for prevention and treatment of depression in menopausal women, including a Salvia miltiorrhiza extract as an active ingredient.

In one embodiment of the present disclosure, the “menopausal women” may be menopausal women naturally occurring due to aging, but is not limited thereto.

In one embodiment of the present disclosure, the “extract” may use at least one solvent of purified water, alcohols having carbon atoms 1 to 4 including methanol, ethanol, propanol, isopropanol, butanol, etc., acetone, ether, benzene, chloroform, ethyl acetate, methylene chloride, hexane, and cyclohexane, but is not limited thereto.

In one embodiment of the present disclosure, the “solvent” may be ethanol at a concentration of 60 to 80 wt %, but is not limited thereto.

In one embodiment of the present disclosure, the “extract” may be extracted at 80 to 90° C. for 5 to 6 hours, filtered and concentrated under reduced pressure, but is not limited thereto.

In one embodiment of the present disclosure, the “extract” may be extracted by repeating the extraction process at 80 to 90° C. for 5 to 6 hours 2 to 5 times, but is not limited thereto.

In one embodiment of the present disclosure, the “depression” may be caused by a decrease in release of the female hormones during menopause, but is not limited thereto.

In one embodiment of the present disclosure, the “composition” may increase the release of serotonin and norepinephrine, but is not limited thereto.

Another aspect of the present disclosure provides a composition for regulating the release of female hormones, including a Salvia miltiorrhiza extract as an active ingredient.

In one embodiment of the present disclosure, the “composition” may decrease the release of follicle-stimulating hormone, but is not limited thereto.

In one embodiment of the present disclosure, the “composition” may increase the release of luteinizing hormone, but is not limited thereto.

Yet another aspect of the present disclosure provides a health functional food composition for prevention and alleviation of depression in menopausal women, including a Salvia miltiorrhiza extract as an active ingredient.

Still another aspect of the present disclosure provides a method for treating depression in menopausal women including administering a pharmaceutical composition including the Salvia miltiorrhiza extract to a subject.

### Advantageous Effects

According to the present disclosure, the Salvia miltiorrhiza extract decreases the release of follicle-stimulating hormone, which is a major symptom in menopausal women, and increases the release of serotonin and norepinephrine which are hormones closely related to depression. Accordingly, the composition including the Salvia miltiorrhiza extract of the present disclosure as an active ingredient can be applied to a pharmaceutical composition for prevention and treatment of depression by regulating the hormones in menopausal women, and can be usefully used as a health functional food composition for prevention and alleviation of depression by regulating the hormones in menopausal women.

### DESCRIPTION OF DRAWINGS

FIG. 1 is a diagram illustrating an effect of regulating female hormone receptors of ingredients included in a Salvia miltiorrhiza extract.

FIG. 2 is a diagram illustrating results of measuring the stability of various ingredients (a total of 5 types) included in the Salvia miltiorrhiza extract by time.

FIG. 3 is a diagram illustrating results of measuring the stability of various ingredients (a total of 5 types) included in the Salvia miltiorrhiza extract by date.

FIG. 4 is a diagram illustrating a regulatory effect of follicle-stimulating hormone (FSH) according to the intake of the Salvia miltiorrhiza extract in a menopausal animal model induced by natural aging.

FIG. 5 is a diagram illustrating a regulatory effect of luteinizing hormone (LH) according to the intake of the Salvia miltiorrhiza extract in a menopausal animal model induced by natural aging.

FIG. 6 is a diagram illustrating a regulatory effect of serotonin according to the intake of the Salvia miltiorrhiza extract in a menopausal animal model induced by natural aging.

FIG. 7 is a diagram illustrating a regulatory effect of norepinephrine according to the intake of the Salvia miltiorrhiza extract in a menopausal animal model induced by natural aging.

FIG. 8 is a diagram illustrating a regulatory effect of serotonin according to the intake of a 70% alcohol extract of Salvia miltiorrhiza at different concentrations in a menopausal animal model induced by natural aging.

FIG. 9 is a diagram illustrating a regulatory effect of norepinephrine according to the intake of a 70% alcohol extract of Salvia miltiorrhiza at different concentrations in a menopausal animal model induced by natural aging.

FIG. 10 is a diagram illustrating an antidepressant effect according to a dose of the 70% alcohol extract of Salvia miltiorrhiza of the present disclosure by a forced swimming measurement method in the menopausal animal model induced by natural aging.

FIG. 11 is a diagram illustrating an antidepressant effect according to a dose of the 70% alcohol extract of Salvia miltiorrhiza of the present disclosure by a tail suspension measurement method in the menopausal animal model induced by natural aging.

### BEST MODE OF THE INVENTION

Hereinafter, an embodiment of the present disclosure will be described in detail with reference to the accompanying drawings. However, the following exemplary embodiments are presented as examples for the present disclosure, and when it is determined that a detailed description of well-known technologies or configurations known to those skilled in the art may unnecessarily obscure the gist of the present disclosure, the detailed description thereof may be omitted, and the present disclosure is not limited thereto. Various modifications and applications of the present disclosure are possible within the description of claims to be described below and the equivalent scope interpreted therefrom.

Terminologies used herein are terminologies used to properly express embodiments of the present disclosure, which may vary according to a user, an operator's intention, or customs in the art to which the present disclosure pertains. Throughout the specification, unless explicitly described to the contrary, when a certain part “comprises” a certain component, it will be understood to imply the inclusion of stated elements but not the exclusion of any other elements.

Throughout this specification, ‘%’ used to indicate the concentration of a specific material is solid/solid (w/w) %, solid/liquid (w/v) %, and liquid/liquid (v/v) %, unless otherwise stated.

All technical terms used in the present disclosure, unless otherwise defined, have the meaning as commonly understood by those skilled in the related art of the present disclosure. In addition, although preferred methods and samples are described herein, similar or equivalent methods and samples thereto are also included in the scope of the present disclosure. The contents of all publications disclosed as references in this specification are incorporated in the present disclosure.

In an aspect, an object of present disclosure is to provide a method for extracting a Salvia miltiorrhiza extract from a pharmaceutical composition for prevention and treatment of depression in menopausal women, including a Salvia miltiorrhiza extract as an active ingredient.

The extract according to the present disclosure may be used what is obtained by extraction and separation from the nature using extraction and separation methods known in the art, and the “extract” defined in the present disclosure refers to what is extracted from Salvia miltiorrhiza using a suitable solvent, and includes all, for example, a crude extract, a polar solvent-soluble extract, or a non-polar solvent-soluble extract. As a suitable solvent for extracting the extract from the Salvia miltiorrhiza, any pharmaceutically acceptable organic solvent may be used, and water or an organic solvent may be used, but is not limited thereto. For example, various solvents such as purified water, alcohols having carbon atoms 1 to 4 including methanol, ethanol, propanol, isopropanol, and butanol, acetone, ether, benzene, chloroform, ethyl acetate, methylene chloride, hexane, and cyclohexane, may be used alone or in combination. As an extraction method, any one of methods such as hot water extraction, chilling extraction, reflux cooling extraction, solvent extraction, steam distillation, ultrasonic extraction, elution, and compression may be selected and used. In addition, a desired extract may also be additionally subjected to a conventional fractionation process or may be purified using a conventional purification method.

There is no limitation on the preparation method of the extract of the present disclosure, and any known method may be used. For example, the extract included in the composition of the present disclosure may be prepared in a powder state by additional processes such as distilling under reduced pressure and freeze-drying or spray-drying of a primary extract extracted by the hot water extraction or solvent extraction method. In addition, a fraction may also be obtained by further purifying the primary extract using various chromatography such as silica gel column chromatography, thin layer chromatography, and high performance liquid chromatography. Accordingly, in the present disclosure, the extract is a concept that includes all extracts, separated compounds, fractioned and purified products obtained in each step of extraction, fractionation, or purification, and dilutions, concentrates, or dried products thereof.

In addition, the Salvia miltiorrhiza extract defined in the present disclosure includes both water-soluble and fat-soluble ingredients, and preferably contains fat-soluble ingredients.

The Salvia miltiorrhiza extract may be a Salvia miltiorrhiza extract soluble in a solvent selected from water, lower alcohols having 1 to 4 carbon atoms such as methanol, ethanol, and butanol or mixed solvents thereof, preferably a mixed solvent of water and ethanol, and more preferably 50 to 100% ethanol. The Salvia miltiorrhiza extract may use a Salvia miltiorrhiza extract obtained by a conventional extraction method.

Specifically, the Salvia miltiorrhiza extract may be obtained by including a first step of obtaining a crude extract through filtration, concentration under reduced pressure, and drying processes after extraction of dried Salvia miltiorrhiza by extraction methods such as chilling extraction, hot water extraction, ultrasonic extraction, reflux cooling extraction, or heating extraction method, preferably reflux cooling extraction, at 10 to 100° C., preferably 30 to 80° C. for 1 to 24 hours, preferably 2 to 12 hours, in a solvent selected from water containing purified water in about 1 to 1000-fold, preferably about to 100-fold volume amount (w/v %) of the weight of dry Salvia miltiorrhiza, lower alcohols having 1 to 4 carbon atoms, such as methanol, ethanol, butanol, isopropanol, and butanol, or mixed solvents thereof; a second step of obtaining a water-insoluble residue by suspending and filtering the crude extract by adding water in a 1 to 10-fold, preferably 3 to 6-fold volume of the weight of the crude extract; a third step of obtaining a filtrate through stirring, dissolving and filtrating processes by adding the residue with the solvent selected from water containing purified water, lower alcohols having 1 to 4 carbon atoms, such as methanol, ethanol, and butanol, or mixed solvents thereof, preferably a mixed solvent of water and ethanol, that is, 20% to 95% ethanol, more preferably 50% to 95% ethanol, much more preferably 70% ethanol; and a step of removing the residual solvent of the filtrate.

The Salvia miltiorrhiza extract obtained in the extraction process may be further subjected to a process such as concentration and drying, and in the drying process, the Salvia miltiorrhiza extract may be obtained in the form of a powdery dry product by freeze-drying. In the case of applying the freeze-drying method in the drying process, there is an advantage of reducing the loss of volatile organic substances in the Salvia miltiorrhiza extract.

In an aspect, an object of the present disclosure is to provide a pharmaceutical composition for prevention and treatment of depression in menopausal women, including a Salvia miltiorrhiza extract as an active ingredient.

As used herein, the term “treatment” refers to all actions that relieve or beneficially change symptoms of depression in menopausal women by administering the composition of the present disclosure. Those of ordinary skill in the art to which the present disclosure pertains will be able to determine the degree of alleviation, enhancement and treatment by knowing the exact criteria of a disease for which the composition of the present disclosure is effective by referring to data presented by the Korean Academy of Medical Sciences, etc.

In one embodiment, the pharmaceutical composition may be one or more formulations selected from the group including oral formulations, external formulations, suppositories, sterile injection solutions and sprays, and injectable formulations are more preferred.

In the present disclosure, the term “prevention” refers to all actions that inhibit or delay the occurrence, spread, and recurrence of depression in menopausal women by administration of the pharmaceutical composition according to the present disclosure.

In the present disclosure, the term “therapeutically effective dose” used in combination with the active ingredients means an amount of a pharmaceutically acceptable salt of the composition effective for preventing or treating a target disease, and the therapeutically effective dose of the composition of the present disclosure may vary depending on many factors, such as a method of administration, a target site, and the condition of a patient. Accordingly, when used in the human body, the dose should be determined as an appropriate amount in consideration of both safety and efficiency. It is also possible to estimate the amount used in humans from the effective dose determined through animal experiments. These matters to be considered when determining the effective dose are described in, for example, Hardman and Limbird, eds., Goodman and Gilman's The Pharmacological Basis of Therapeutics, 10th ed. (2001), Pergamon Press; and E. W. Martin ed., Remington's Pharmaceutical Sciences, 18th ed. (1990), Mack Publishing Co.

The pharmaceutical composition of the present disclosure is administered in a pharmaceutically effective dose. As used herein, the term “pharmaceutically effective dose” refers to an amount enough to treat diseases at a reasonable benefit/risk ratio applicable to medical treatment and enough to not cause side effects. An effective dose level may be determined according to a health condition of a patient, a disease type, severity, drug activity, sensitivity to drug, an administration method, an administration time, an administration route and excretion rate, a treatment period, factors including drugs used in combination or concurrently, and other factors well-known in medical fields. The composition of the present disclosure may be administered as an individual therapeutic agent or in combination with other therapeutic agents, and may be administered sequentially or simultaneously with existing therapeutic agents, and may be administered singly or multiply. It is important to administer an amount capable of obtaining a maximum effect with a minimal amount without side effects by considering all the factors, which may be easily determined by those skilled in the art.

In the present disclosure, the “subject” is not particularly limited as long as the subject is any object for the purpose of preventing or treating menopausal depression, and includes animals including humans, such as non-primates (e.g., cow, pig, horse, cat, dog, rats and mice) and primates (e.g., monkey such as Cynomolgus monkeys and chimpanzee). In some cases, the subject may be effective for human females, and in some cases, the subject may be for a subject other than humans.

The pharmaceutical composition of the present disclosure may further include a pharmaceutically acceptable additive. At this time, the pharmaceutically acceptable additive may use starch, gelatinized starch, microcrystalline cellulose, lactose, povidone, colloidal silicon dioxide, calcium hydrogen phosphate, lactose, mannitol, syrup, gum arabic, pregelatinized starch, corn starch, powdered cellulose, hydroxypropyl cellulose, Opadry, sodium starch glycolate, lead carnauba, synthetic aluminum silicate, stearic acid, magnesium stearate, aluminum stearate, calcium stearate, sucrose, dextrose, sorbitol, talc and the like. The pharmaceutically acceptable additives according to the present disclosure is preferably included in an amount of 0.1 parts by weight to 90 parts by weight based on the composition, but is not limited thereto.

The composition of the present disclosure may include a carrier, a diluent, an excipient, or a combination of two or more thereof, which are commonly used in biological agents. The pharmaceutically acceptable carrier is not particularly limited as long as the pharmaceutically acceptable carrier is suitable for in vivo delivery of the composition, and may be used by combining, for example, compounds described in Merck Index, 13th ed., Merck & Co. Inc., saline, sterile water, a Ringer's solution, buffered saline, a dextrose solution, a maltodextrin solution, glycerol, ethanol, and one or more of these ingredients, and if necessary, other conventional additives such as an antioxidant, a buffer, and a bacteriostat may be added. In addition, the pharmaceutical composition may be prepared in injectable formulations such as an aqueous solution, a suspension, and an emulsion, pills, capsules, granules, or tablets by further adding a diluent, a dispersant, a surfactant, a binder, and a lubricant. Furthermore, the pharmaceutical composition may be preferably prepared according to each disease or ingredients using as a suitable method in the art or a method disclosed in Remington's Pharmaceutical Science (Mack Publishing Company, Easton PA, 18th, 1990).

The composition of the present disclosure may be administered parenterally (e.g., applied with injectable formulations intravenously, subcutaneously, intraperitoneally or topically) or orally according to a desired method, and the range of the dose may vary depending on the body weight, age, sex, and health condition of a patient, a diet, an administration time, an administration method, an excretion rate, the severity of a disease, and the like. A daily dose of the composition according to the present disclosure is to 10 mg/ml, preferably 0.0001 to 5 mg/ml, and more preferably administered once to several times a day.

Liquid formulations for oral administration of the composition of the present disclosure correspond to suspensions, internal solutions, emulsions, syrups, etc., and may include various excipients, such as wetting agents, sweeteners, fragrances, and preservatives, in addition to water and liquid paraffin, which are commonly used simple diluents. Formulations for parenteral administration include sterilized aqueous solutions, non-aqueous solvents, suspensions, emulsions, lyophilized agents, and suppositories.

The pharmaceutical composition of the present disclosure may be administered together with an existing antidepressant composition to increase an antidepressant effect. Combined administration may be performed concurrently or sequentially with existing antidepressant compositions.

In one embodiment of the present disclosure, in an antidepressant efficacy verification experiment for a Salvia miltiorrhiza extract in a natural aging-induced menopausal animal model, it was confirmed that there was an antidepressant effect when a 70% alcohol extract of Salvia miltiorrhiza was administered to an antidepressant refractory animal model that had an insufficient antidepressant effect on an antidepressant benzodiazepine administered group (BZ) or an imipramine administered group (I). Accordingly, the Salvia miltiorrhiza extract of the present disclosure may be administered to patients with resistance to benzodiazepine or imipramine, and may be used to treat depression that is resistant to the drug.

In an aspect, an object of the present disclosure is to provide a health functional food composition for prevention and alleviation of depression in menopausal women, including a Salvia miltiorrhiza extract as an active ingredient.

When the Salvia miltiorrhiza extract of the present disclosure is used as the health functional food composition, the Salvia miltiorrhiza extract may be added as it is or used together with other foods or food ingredients, and appropriately used according to a conventional method. The composition may include food acceptable supplement additives in addition to the active ingredients, and the mixing amount of the active ingredients may be appropriately determined depending on the purpose of use (prevention, health or therapeutic treatment).

The term “food supplement additive” used in the present disclosure means a component that may be supplementally added to food, and may be appropriately selected and used by those skilled in the art as being added to prepare a health functional food of each formulation. Examples of the food supplement additives include various nutrients, vitamins, minerals (electrolytes), flavors such as synthetic and natural flavors, colorants and fillers, pectic acid and salts thereof, alginic acid and salts thereof, organic acids, protective colloidal thickeners, pH adjusters, stabilizers, preservatives, glycerin, alcohols, carbonation and agents used in carbonated drinks, but the types of food supplement additives of the present disclosure are not limited by the above examples.

The food composition of the present disclosure may include a health functional food. The term “health functional food” used herein refers to food prepared and processed in the form of tablets, capsules, powders, granules, liquids and pills by using raw materials or ingredients having functionalities useful to the human body. Here, the ‘functionality’ refers to regulating nutrients to the structure and function of the human body or to obtaining effects useful for health applications such as physiological action. The health functional food of the present disclosure may be prepared by methods to be commonly used in the art and may be prepared by adding raw materials and ingredients which are commonly added in the art in preparation. In addition, the formulations of the health functional food may also be prepared without limitation as long as the formulation is recognized as a health functional food. The food composition of the present disclosure may be prepared in various types of formulations, and unlike general drugs, the food composition has an advantage that there is no side effect that may occur when taking a long-term use of the drug by using the food as a raw material, and has excellent portability, so that the health functional food of the present disclosure may be taken as supplements to enhance an anti-inflammatory effect.

In addition, there is no limitation in a type of health food in which the composition of the present disclosure may be used. In addition, the composition including the Salvia miltiorrhiza extract of the present disclosure as an active ingredient may be prepared by mixing known additives with other suitable auxiliary ingredients that may be contained in the health functional food according to the selection of those skilled in the art. Examples of foods to be added include meat, sausage, bread, chocolate, candy, snacks, confectionery, pizza, ramen, other noodles, gum, dairy products including ice cream, various soups, beverages, tea, drinks, alcoholic beverages, and vitamin complexes, and may be prepared to be added to extract, tea, jelly, juice, and the like prepared by using the extract according to the present disclosure as a main ingredient.

Since Salvia miltiorrhiza Bunge (SM) of the Salvia miltiorrhiza extract of the present disclosure is a medicinal herb that has been widely used in oriental medicine since ancient times and a perennial plant belonging to the dicotyledoneae, Tubiflorae, Labiatae, when used as a pharmaceutical composition or health functional food composition, there are side effects less than those of general synthetic compounds, so that it may be safely included in the pharmaceutical composition and the health functional food compositions to be usefully used.

Hereinafter, the present disclosure will be described in more detail with reference to the following Example embodiments. However, the following Example embodiments are only intended to embody the contents of the present disclosure, and the present disclosure is not limited thereto.

### <Example Embodiment 1> Preparation of Salvia miltiorrhiza Extract

1-1. Preparation of Hot Water Extract of Salvia miltiorrhiza

Dried Salvia miltiorrhiza was added with distilled water in a 25-fold volume (w/v %) of the weight of Salvia miltiorrhiza and heated at 100° C. for 6 hours to obtain an extract by hot water extraction, and the obtained extract was filtered, concentrated and dried at 50° C. to obtain a hot water extract of Salvia miltiorrhiza.

1-2. Preparation of Alcohol Extract of Salvia miltiorrhiza

Dried Salvia miltiorrhiza was added with 70% alcohol in a 10-fold volume (w/v %) of the weight of Salvia miltiorrhiza, and subjected to primary extraction at 80° C. for 5 hours at 1.5 atm or less, and then filtered. The obtained extract was concentrated at 65° C. or less using a decompressed concentrator to obtain an alcohol extract of Salvia miltiorrhiza.

1-3. Preparation of Distilled Extract of Salvia miltiorrhiza (Essential Oil by Distillation Extraction Method)

Salvia miltiorrhiza was added into a distillation tank of an essential oil extraction device, and steam was generated by operating a boiler of a steam generator. Thereafter, the stream passed through the distillation tank containing Salvia miltiorrhiza. At this time, the temperature in the distillation tank was 110° C. and the process was performed at atmospheric pressure. While passing through the distillation tank, the stream mixed with an essential oil ingredient was condensed through a cooling device to obtain a light yellow distilled extract of Salvia miltiorrhiza using an oil-water separator.

### <Example Embodiment 2> Evaluation of Ingredients Included in Salvia Miltiorrhiza Extract

2-1. Evaluation of Female Hormone Regulating Ability of Ingredients Included in Salvia miltiorrhiza Extract

Ingredients included in the Salvia miltiorrhiza extract obtained in Example embodiment 1 were confirmed, and in ingredients cryptotanshinone, danshensu, isotanshinone, salvianolic acid B, and tanshinone IIA, female hormone regulating ability was confirmed. An MCF-7 BUS cell line used in the experiment was received from Tufts University in the United States, and incubated in a CO₂ incubator maintained at 37° C. and 5% CO₂ by using a Dulbecco modified eagle medium (DMEM) (Gibco, USA) added with 10% fetal bovine serum (Gibco, USA) and 1% penicillin/streptomycin (Gibco, USA).

In the experiment, in order to evaluate the female hormone regulatory ability, the sensitivity to estrogen, one of the female hormones, was measured using the MCF-7 BUS cell line, and first, the MCF-7 BUS cell line was dispensed at 1×10⁵ cells/well in a 60 mm culture dish, and then incubated in a CO₂ incubator maintained at 37° C. and 5% CO₂.

After incubation for 24 hours, each ingredient was treated at a concentration of 10 μg/mL, and 17β-Estradiol (E2) was treated with 10⁻¹⁰ M as a positive control. After 72 hours, mRNA isolated using a PureLink™ RNA mini kit (Ambion, USA) was converted to cDNA using a PrimeScript™ II 1st strand cDNA synthesis kit (Takara, Japan), and real-time PCR (RT-PCR, Stratagene, USA) was performed using Syber Green (Takara, Japan) as a material binding to the amplified DNA to exhibit fluorescence.

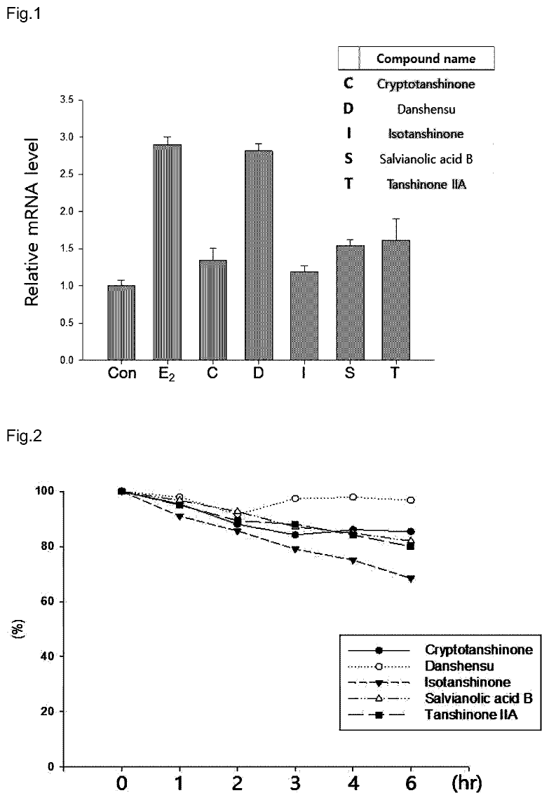

At this time, each primer was prepared by Takara, and ER was prepared with Forward primer: CGCTACTGTGCAGTGTGCAAT (SEQ ID NO: 1) and Reverse primer: CCTCACAGGACCAGACTCCATAA (SEQ ID NO: 2). The PCR conditions were predenatured at 95° C. for 30 seconds in the first step, and repeated total 40 times at for 30 seconds and 60° C. for 1 minute in the next step. A CT value obtained through RT-PCR was converted into ΔCT, and this ΔCT value was analyzed through a change rate of an experimental group with respect to the control group, which was illustrated in FIG. 1. As illustrated in FIG. 1, among various ingredients included in the Salvia miltiorrhiza extract, danshensu exhibited the most remarkable female hormone receptor regulating effect.

2-2. Evaluation of Stability of Ingredients Included in Salvia miltiorrhiza Extract

When the Salvia miltiorrhiza extract was stored at 95° C. for 0 hour, 1 hour, 2 hours, 3 hours, 4 hours and 6 hours, in order to confirm whether the stability of 5 types of ingredients of which the female hormone regulatory ability has been confirmed in Example embodiment 2-1 above was maintained, the ingredient contents of the stored samples were analyzed by time. In addition, when the Salvia miltiorrhiza extract was stored at room temperature for 1 day, 2 days, 3 days, 4 days and 5 days, the stability of the ingredients was confirmed through analysis of the contents of 5 types of ingredients included in the Salvia miltiorrhiza extract.

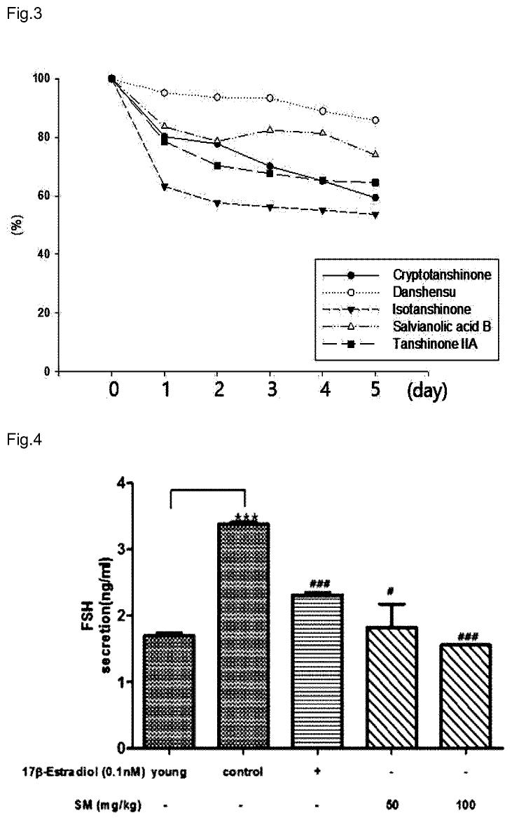

As a result, as illustrated in FIGS. 2 and 3, among the ingredients included in the Salvia miltiorrhiza extract, danshensu was not denaturated and had high stability, despite long-term storage, so that the effect thereof may be maintained.

In addition, results of comparing the content of each ingredient included in the Salvia miltiorrhiza extract at various concentrations (0, 50, 70, and 100% ethanol) and the content after 5 days of storage at room temperature were shown together in Table 1 below.

TABLE 1

| | Salvia miltiorrhiza extract | | | | After 5 days of storage at room | | | |
|---|---|---|---|---|---|---|---|---|
| | (EtOH %) | | | | temperature of extract (EtOH %) | | | |
| Content (mg/g) | 0 | 50 | 70 | 100 | 0 | 50 | 70 | 100 |
| C (Cryptotanshinone) | 0.11 | 0.63 | 3.61 | 3.22 | 0.00 | 0.24 | 0.11 | 0.12 |
| D (Danshensu) | 0.34 | 1.28 | 25.17 | 18.32 | 0.32 | 1.22 | 24.19 | 17.33 |
| I (Isotanshinone) | 2.22 | 12.31 | 14.23 | 12.22 | 0.99 | 5.62 | 12.11 | 10.33 |
| S (Salvianolic acid B) | 21.2 | 38.23 | 33.1 | 20.3 | 18.33 | 26.21 | 23.21 | 15.27 |
| T (Tanshinone IIA) | 1.33 | 5.21 | 7.22 | 8.33 | 0.65 | 4.11 | 4.33 | 5.24 |
| ER expression (%) | 23.7 | 95.2 | 176.6 | 132.3 | 20.5 | 87.2 | 166.4 | 129.1 |

(C, Cryptotanshinone; D, Danshensu; I, Isotanshinone; S, Salvianolic acid B; T, Tanshinone IIA; E2: female hormone, ER: female hormone receptor, ER expression of E2 = based on 100%)

Through the results, the following experiment was performed by using a 70% alcohol extract of Salvia miltiorrhiza in which the stability of raw materials was excellent so that the efficacy was continuously maintained during a long-term storage, and the content of each ingredient included in the Salvia miltiorrhiza extract was highest.

### <Example Embodiment 3> Hormone Release Regulatory Effect of Salvia Miltiorrhiza Extract

3-1. Regulation of Release of Female Hormone in Natural Aging-Induced Menopausal Animal Model

A 70% alcohol extract of Salvia miltiorrhiza was orally administered to 52-week-old female B6 mice at doses of 50 and 100 mg/kg for 10 weeks. At this time, 5- and 52-week-old female B6 mice untreated with a Salvia miltiorrhiza alcohol extract were used as a control group. In addition, as a positive control group, 0.1 mg/kg 17β-Estradiol (E2) was administered intraperitoneally, and after administration was completed, blood was collected and centrifuged to obtain serum. The release amounts of follicle-stimulating hormone (FSH) and luteinizing hormone (LH) in each experimental group were measured using an ELISA kit.

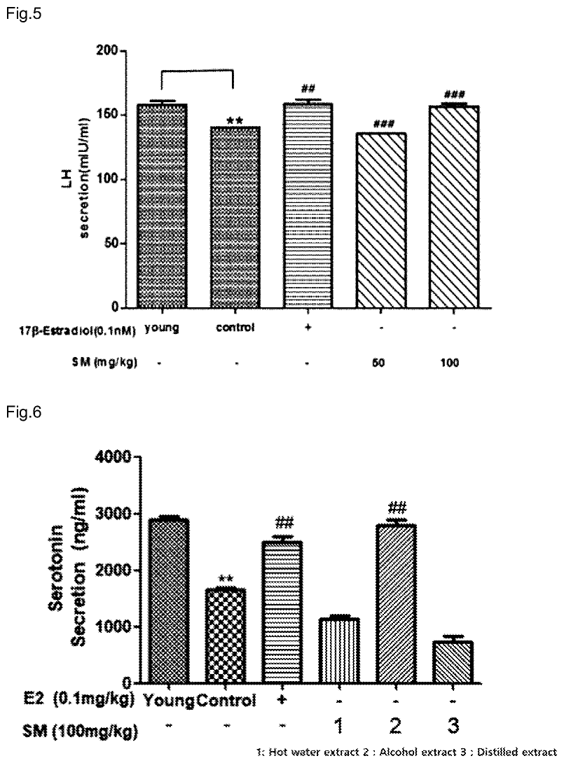

As a result, as illustrated in FIG. 4, a group administered with 50 mg/kg of the 70% alcohol extract of Salvia miltiorrhiza released follicle-stimulating hormone (FSH) at a level similar to that of a 5-week-old female mouse group, and compared to the control group, the release amount of follicle-stimulating hormone was significantly reduced, and a group administered with 100 mg/kg of the Salvia miltiorrhiza extract effectively reduced the release of the follicle-stimulating hormone. In addition, as illustrated in FIG. 5, the group administered with 100 mg/kg of the 70% alcohol extract of Salvia miltiorrhiza increased the release amount of luteinizing hormone (LH) at a level similar to that of the 5-week-old female mouse group compared to the control group.

3-2. Regulation of Release of Serotonin and Norepinephrine in Natural Aging-Induced Menopausal Animal Model

A hot water extract of Salvia miltiorrhiza, a 70% alcohol extract of Salvia miltiorrhiza, and a distilled extract of Salvia miltiorrhiza obtained in Example embodiment were orally administered to a menopausal animal model induced by natural aging to examine the release amounts of serotonin and norepinephrine, which were depression-related hormones in the menopausal women.

In Example embodiment, 52-week-old female mice untreated with the Salvia miltiorrhiza extract were used as a control group, and each Salvia miltiorrhiza extract was orally administered at a dose of 100 mg/kg for 10 weeks. Also, as a positive control group, 17beta-estradiol (E2) was intraperitoneally administered. The hormone release amount was measured using an ELISA kit after separating the serum from the experimental animals.

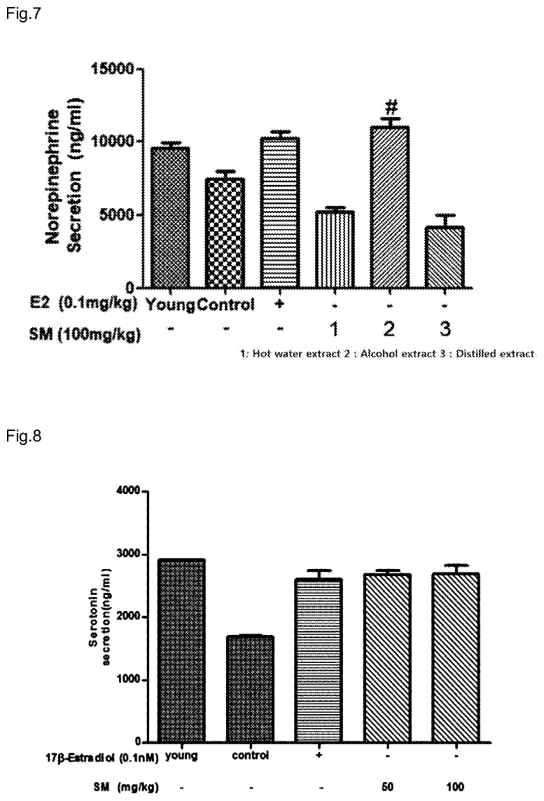

As a result, as illustrated in FIG. 6, the hot water extract and the distilled extract of Salvia miltiorrhiza decreased the release of serotonin, but the 70% alcohol extract of Salvia miltiorrhiza increased the release of serotonin. In addition, as illustrated in FIG. 7, the hot water extract and the distilled extract of Salvia miltiorrhiza decreased the release of norepinephrine, but the 70% alcohol extract of Salvia miltiorrhiza increased the release of norepinephrine. These results showed that the 70% alcohol extract of Salvia miltiorrhiza had an effect of alleviating and treating depression by increasing the release amount of serotonin and norepinephrine, which were hormones closely related to depression.

3-3. Regulation of Release of Serotonin and Norepinephrine According to Concentration of 70% Alcohol Extract of Salvia miltiorrhiza in Natural Aging-Induced Menopausal Animal Model

According to the results of Example embodiment, the effect of regulating the release of serotonin and norepinephrine was examined according to the concentrations (50 and 100 mg/kg) of the 70% alcohol extract of Salvia miltiorrhiza.

As a control group, 5-week-old and 52-week-old female B6 mice untreated with the 70% alcohol extract of Salvia miltiorrhiza were used, and the 70% alcohol extract of Salvia miltiorrhiza was orally administered to 52-week-old female B6 mice at doses of mg/kg and 100 mg/kg for 10 weeks. As a positive control group, 0.1 mg/kg 17β-Estradiol (E2) was administered intraperitoneally, and after administration was completed, blood was collected and centrifuged to obtain serum. The release amounts of serotonin and norepinephrine in each experimental group were measured using an ELISA kit.

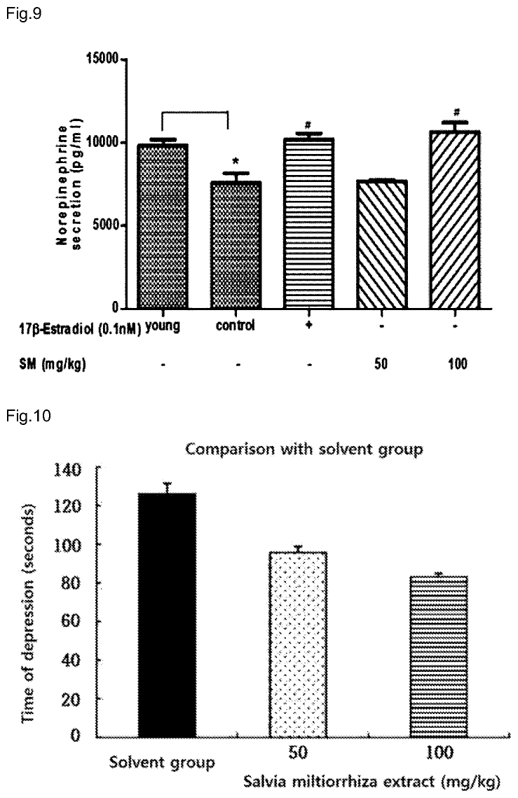

As a result, as illustrated in FIG. 8, it was confirmed that the release amount of serotonin was increased in both groups administered with 50 mg/kg of the 70% alcohol extract of Salvia miltiorrhiza and 100 mg/kg of the 70% alcohol extract of Salvia miltiorrhiza as compared with a 52-week-old female B5 mouse control group which was a menopausal animal model, and the release amount of serotonin was restored to a level similar to that of a 5-week-old female B6 mouse control group, which was a premenopausal animal model. In addition, as illustrated in FIG. 9, it was confirmed that in the case of the release amount of norepinephrine, in the group administered with 50 mg/kg of the 70% alcohol extract of Salvia miltiorrhiza, there was no difference from the control group of the 52-week-old female B5 mice as the menopausal animal model, but in the group administered with 100 mg/kg of the 70% alcohol extract of Salvia miltiorrhiza, the release amount of norepinephrine was increased at a higher level than the 5-week-old female B6 mouse control group, which was the premenopausal animal model. These results showed that 100 mg/kg of the 70% alcohol extract of Salvia miltiorrhiza had a greater effect of alleviating and treating depression than 50 mg/kg of the 70% alcohol extract of Salvia miltiorrhiza by increasing the release amounts of serotonin and norepinephrine, which were hormones closely related to depression.

According to Example embodiments above, in the present disclosure, it was confirmed that the Salvia miltiorrhiza extract effectively increased the release of follicle-stimulating hormone and the release amount of luteinizing hormone (LH) in the menopausal animal model induced by natural aging. In addition, it was confirmed that the Salvia miltiorrhiza extract increased the release amounts of serotonin and norepinephrine, which were hormones closely related to depression. It is meant that that the Salvia miltiorrhiza extract may alleviate and treat depression caused by menopause naturally occurring by aging. Therefore, the Salvia miltiorrhiza extract may be used as a composition for preventing, alleviating, and treating depression in menopausal women.

### <Example Embodiment 4> Verification of Antidepressant Efficacy of Salvia Miltiorrhiza Extract in Natural Aging-Induced Menopausal Animal Model

### 4-1. Antidepressant Effect of Salvia miltiorrhiza Extract by Forced Swimming Measurement Method

A forced swimming measurement method was one of animal testing methods for examining the motor activity and depression level of laboratory animals, and in a 52-week-old female B5 mouse which was a menopausal animal model, 20 cm of water at 24±1° C. was filled in a cylinder with a diameter of 13 cm and a depth of 30 cm to prevent the mouse's tail from being in contact with the bottom of the cylinder. 50 mg/kg and 100 mg/kg of the 70% alcohol extract of Salvia miltiorrhiza obtained in Example embodiment 1 were administered to the mouse, respectively, and after 1 hour, the mouse was put in the cylinder and observed for 6 minutes. The control group was treated in the same manner as above, except for administering physiological saline. During observation, the immobility time at which the mouse remained still without movement with only its head above water for the remaining 4 minutes except for the first 2 minutes was measured as the time at which depression was shown.

As a result, as illustrated in FIG. 10, in the forced swimming measurement method, it was shown that the experimental groups administered with 50 mg/kg and 100 mg/kg of the 70% alcohol extract of Salvia miltiorrhiza alleviated the antidepressant effect by 23.7% and 34.2%, respectively, compared to the control group. The results indicated that the administration of the 70% alcohol extract of Salvia miltiorrhiza of the present disclosure had antidepressant activity.

4-2. Antidepressant Effect of Salvia miltiorrhiza Extract by Tail Suspension Measurement Method

The tail suspension measurement method of the 52-week-old female B5 mouse as a menopausal animal model was performed by suspending the mouse by attaching 1 to 2 cm of the tip of the mouse's tail with a strong adhesive tape at the edge of a height of 80 cm. The mouse was orally administered with 50 mg/kg and 100 mg/kg of the 70% alcohol extract of Salvia miltiorrhiza obtained in Example embodiment 1 or physiological saline, respectively, and administered intraperitoneally with 10 mg/kg of imipramine (I) as an antidepressant, and after 1 hour, the mouse was suspended and observed for 6 minutes. During the observation, the time at which the mouse stayed still without movement was measured based on the remaining 5 minutes except for the first 1 minute.

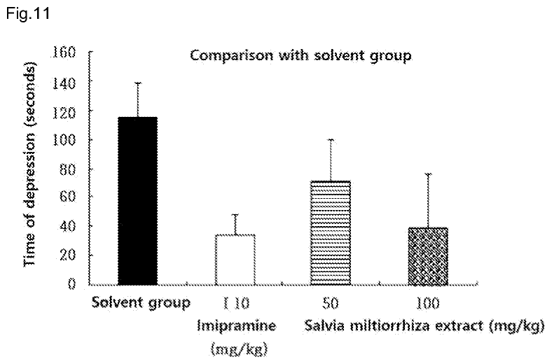

As a result, as illustrated in FIG. 11, in the tail suspension measurement method, compared to the control group, the groups administered with 50 mg/kg and 100 mg/kg of the 70% alcohol extract of Salvia miltiorrhiza had an excellent antidepressant effect of 38.3% and 66.1% which were almost similar levels to the group administered with imipramine (I). Accordingly, the results indicated that the administration of the 70% alcohol extract of Salvia miltiorrhiza of the present disclosure had antidepressant activity.

### <Example Embodiment 5> Acute Toxicity Study

25±5 g of ICR-based mice and 230±10 g of specific pathogen-free (SPF) Sprague-Dawley (Biogenomics) rats were divided into 4 groups of 5 animals, and intraperitoneally administered with the 70% alcohol extract of Salvia miltiorrhiza of Example embodiment 1 of the present disclosure at doses of 50 mg/kg and 100 mg/kg, respectively, and toxicity was observed for 24 hours. As a result of the experiment, no case of death could be observed in all the 4 groups, and there were no particular symptoms in appearance different from those of the control group in terms of weight gain, feed intake, and the like. Therefore, it was confirmed that the Salvia miltiorrhiza extract of the present disclosure was a safe drug.

1. A pharmaceutical composition for prevention and treatment of depression in naturally-occurring menopausal women, comprising:
a Salvia miltiorrhiza extract as an active ingredient,
wherein the Salvia miltiorrhiza extract is extracted using ethanol at a concentration of 60 to 80 wt %, and the composition increases the release of serotonin and norepinephrine.

2. The pharmaceutical composition for prevention and treatment of depression in naturally-occurring menopausal women of claim 1, wherein the extract is extracted at 80 to for 5 to 6 hours, and then filtered and concentrated under reduced pressure.

3. The pharmaceutical composition for prevention and treatment of depression in naturally-occurring menopausal women of claim 2, wherein the extract is extracted by repeating the extraction process at 80 to 90° C. for 5 to 6 hours 2 to 5 times.

4. The pharmaceutical composition for prevention and treatment of depression in naturally-occurring menopausal women of claim 1, wherein the depression in the naturally-occurring menopausal women is caused by a decrease in release of serotonin and norepinephrine during menopause.

5. A health functional food composition for prevention and alleviation of depression in naturally-occurring menopausal women, comprising:
a Salvia miltiorrhiza extract as an active ingredient,
wherein the Salvia miltiorrhiza extract is extracted using ethanol at a concentration of 60 to 80 wt %, and the composition increases the release of serotonin and norepinephrine.

6. A method for treating depression in naturally occurring menopausal women, comprising:
administering the pharmaceutical composition of claim 1 to a subject in need thereof.

7. A method for treating depression in naturally occurring menopausal women, comprising:
administering the pharmaceutical composition of claim 2 to a subject in need thereof.

8. A method for treating depression in naturally occurring menopausal women, comprising:
administering the pharmaceutical composition of claim 3 to a subject in need thereof.

9. A method for treating depression in naturally occurring menopausal women, comprising:
administering the pharmaceutical composition of claim 4 to a subject in need thereof.


SEQUENCE LISTING (2 sequences)

SEQ ID NO: 1 - DNA, 21 bp, Artificial Sequence
  Forward Primer
cgctactgtgcagtgtgcaat

SEQ ID NO: 2 - DNA, 23 bp, Artificial Sequence
  Reverse Primer
cctcacaggaccagactccataa